(19) United States
(12) Patent Application Publication
(10) Pub. No.: US 2018/0190374 A1
(43) Pub. Date: Jul. 5, 2018

(54) METHODS OF IMPROVING PATIENT COMPLIANCE TO TREAT CONTRAST-INDUCED INJURY

(71) Applicant: Saghmos Therapeutics, Inc., Old Greenwich, CT (US)

(72) Inventors: Anna KAZANCHYAN, White Plains, NY (US); Shalini CORNELIO, Holmdel, NJ (US)

(21) Appl. No.: 15/861,173

(22) Filed: Jan. 3, 2018

Related U.S. Application Data
(60) Provisional application No. 62/441,738, filed on Jan. 3, 2017.

(51) Int. Cl. G16H 20/10; A61J 7/00; A61J 7/04

(52) U.S. Cl. CPC G16H 20/10; A61J 7/0084; G07F 9/02; A61J 7/0454; A61J 7/0418

(57) ABSTRACT

Methods of patient compliance using a dosage distribution system to prevent contrast-induced acute injury by administering an inhibitor of fatty acid oxidation to a patient in need thereof. Also provided are methods involving use of dosage distribution system with trimetazidine or pharmaceutically acceptable salts thereof for the prevention and/or treatment of contrast-induced acute kidney injury in a subject in need thereof.

### CROSS-REFERENCE TO RELATED APPLICATION

This application claims the benefit of, and priority to, U.S. Provisional Patent Application No. 62/441,738, filed on Jan. 3, 2017, the contents of which are hereby incorporated herein by reference in its entirety.

### TECHNICAL FIELD

Methods of improving patient compliance using a a dosage distribution system to prevent contrast-induced acute injury by administering an inhibitor of fatty acid oxidation to a patient in need thereof.

### BACKGROUND

Non-compliance of patients to drug regimens prescribed by their physicians results in increased cost of medical care and higher complication rates. Non-compliance refers to the failure to take a prescribed dosage of medicine at the prescribed time which results in undermedication or overmedication. Non-compliance is a powerful confounder of evidence-based practice and can affect daily patient management, resulting in inappropriate therapeutic escalation with greater costs and potential for harm. Moreover, the impact of non-compliance is medication-dependent, patient-specific and therefore must be defined and measured in the context of a particular therapy. Accordingly, non-compliance is complex, remains difficult to control, and the ability to identify its presence accurately remains limited. There is a need in the art for a method to improve patient compliance which provides simple monitoring of medication dosing which is non-invasive, reliable, and straightforward to use.

Contrast-induced acute kidney injury (CI-AKI) (also known as contrast-induced nephropathy) is an abrupt deterioration in renal function that can be associated with use of contrast medium. A contrast medium (or contrast agent) is a substance used to enhance the contrast of structures or fluids within the body in medical imaging. Kidney injury may be associated with a sharp decrease in kidney function over a period of 48-72 hours, potentially leading to increased mortality, re-infarction and higher rates of major adverse cardiovascular events (MACE) over 1 year. The symptoms can be similar to those of kidney disease, which include feeling more tired, poor appetite, swelling in the feet and ankles, puffiness around the eyes, or dry and itchy skin. In some cases, this can lead to serious kidney problems and possible heart and blood vessel problems.

A number of therapies for kidney injury have been investigated, including the use of statins, bicarbonate, N-acetylcysteine, ascorbic acid, theophylline and aminophylline, vasodilators, forced diuresis, and renal replacement therapy. Trimetazidine (1-2,3,4-trimethoxybenzyl)piperazine) has been used for angina pectoris and has been marketed outside of the United States in over 90 countries for over 35 years. Trimetazidine, however, has never been approved in the United States for any indication. It was developed by Les Laboratoires Servier (France) and was first authorized in France in 1978. Three pharmaceutical forms are available in Europe: 20 mg tablet, 20 mg/ml oral solution and 35 mg modified release tablets (MR) under the brand names Vastarel® and Vastarel MR®, and is also available as a generic or branded generic with various names in over 90 countries. Trimetazidine has been described as the first cytoprotective anti-ischemic agent that improves myocardial glucose utilization through inhibition of fatty acid metabolism. Trimetazidine has been tested for the prevention of contrast-induced nephropathy in patients, e.g., with high serum creatinine levels undergoing coronary angiography/angioplasty. For example, four investigator-sponsored Phase 2 clinical trials have tested trimetazidine for prevention of CI-AKI. Results of these trials were published in peer-reviewed journals and demonstrated that trimetazidine was more effective than isotonic saline in reducing the risk of contrast-induced nephropathy (CIN). See Onbasili A O, et al., HEART, 2007. 93:698-702, Rahman M M, et al., MYMENSINGH MED J, 2012. 21 (2):292-299, Shehata M, Am. J Cardiol, 2014. 114:389-394, and Liu W, et al., Am. J. Med Sci, 2015. 350:398-402. Finding novel methods to help patients improve compliance to preventive drug therapies remains a significant unmet medical need. Moreover, there remains a need for improved methods of preventing contrast-induced acute kidney injury. Accordingly, methods to improve patient compliance that also prevent contrast-induced acute kidney injury (CI-AKI) has enormous potential to improve health outcomes.

### SUMMARY

In embodiments, methods are provided for preventing and/or treating contrast-induced acute kidney injury which include administering to a patient in need thereof a dosage distribution system including a container for storing a dosage form and a sensor for processing signals wherein the dosage form includes an inhibitor of fatty acid oxidation. In embodiments, methods are provided for preventing and/or treating contrast-induced acute kidney injury including administering to a patient in need thereof a dosage form that includes trimetazidine or a pharmaceutically acceptable salt thereof. For example, methods are provided for preventing contrast-induced acute kidney injury in a patient undergoing a cardiac procedure that requires administration of contrast media for imaging. In embodiments, methods are provided for preventing and/or treating contrast-induced acute kidney injury including administering to a patient in need thereof a dosage form that includes trimetazidine or a pharmaceutically acceptable salt thereof in an amount of more than 20 mg. In embodiments, methods are provided for preventing and/or treating contrast-induced acute kidney injury including administering to a patient in need thereof a dosage form that includes trimetazidine or a pharmaceutically acceptable salt thereof in an amount of more than 35 mg. In embodiments, methods are provided for preventing and/or treating contrast-induced acute kidney injury which include administering to a patient in need thereof etomoxir, oxfenicine, perhexiline, mildronate, or ranolazine or a pharmaceutically acceptable salt of any of the preceding.

In embodiments, the patient is administered an inhibitor of fatty acid oxidation prior to undergoing a cardiac procedure. In embodiments, the patient is administered trimetazidine, etomoxir, oxfenicine, perhexiline, mildronate, or ranolazine or a pharmaceutically acceptable salt of any of the preceding, prior to undergoing a cardiac procedure. In embodiments, the patient is administered trimetazidine or a pharmaceutically acceptable salt thereof prior to undergoing a cardiac procedure. For example, the patient may be administered trimetazidine or a pharmaceutically acceptable salt thereof for more than, e.g., 24, 48, or 72 hours, prior to undergoing a cardiac procedure. In other examples, the patient in need thereof is administered trimetazidine or a pharmaceutically acceptable salt thereof for at least 96 hours prior to undergoing a cardiac procedure. In embodiments, trimetazidine or a pharmaceutically acceptable salt thereof may be administered once a day. In embodiments, trimetazidine or a pharmaceutically acceptable salt thereof may be administered twice daily. In embodiments, trimetazidine or a pharmaceutically acceptable salt thereof may be administered three times daily. In embodiments, the trimetazidine or a pharmaceutically acceptable salt thereof may be administered four times daily.

In embodiments, the patient may be administered an inhibitor of fatty acid oxidation for more than, e.g., 30, 45, 60, 75, or 90 days, after undergoing a cardiac procedure. In embodiments, the patient may be administered trimetazidine or a pharmaceutically acceptable salt thereof for more than, e.g., 30, 45, 60, 75, or 90 days, after undergoing a cardiac procedure. For example, the patient may be administered trimetazidine or a pharmaceutically acceptable salt thereof twice daily for more than 60 days after undergoing a cardiac procedure. In other examples, the patient may be administered trimetazidine or a pharmaceutically acceptable salt thereof twice daily for a total of 90 days.

In embodiments, the patient may be administered an inhibitor of fatty acid oxidation three times daily for more than 48 hours prior to undergoing a cardiac procedure and administered trimetazidine or a pharmaceutically acceptable salt thereof twice daily for more than 60 days after undergoing the cardiac procedure. In embodiments, the patient may be administered trimetazidine or a pharmaceutically acceptable salt thereof three times daily for more than 48 hours prior to undergoing a cardiac procedure and administered trimetazidine or a pharmaceutically acceptable salt thereof twice daily for more than 60 days after undergoing the cardiac procedure.

### DETAILED DESCRIPTION

Described herein are systems for coordinating the administration of medication regimens away from the support system of a hospital or pharmacy, and without the day-to-day supervision of medical personnel. The methods provide for preventing and/or treating contrast-induced acute kidney injury which include administering to a patient in need thereof a dosage distribution system including a container for storing a dosage form and a sensor for processing signals wherein the dosage form includes an inhibitor of fatty acid oxidation. The methods protect the user from overdosage and underdosage, and provide a method of ensuring patient compliance. In embodiments, the dosage distribution system may alert the patient to take a dose of medication and gathers compliance data. In embodiments, the compliance data may be accessible to the patient, a physician, a parent or other caregiver via a network database.

In embodiments the dosage distribution system includes a notification system. For example, the dosage distribution system may include a sensor in each container that stores a dosage form, and the sensor detects status information, e.g., the presence or absence of a tablet in the container and/or the number of tablets in the container. The dosage distribution system may also have a communication circuit, which can be wirelessly connected to exchange data with a device or an Internet-based service. The dosage distribution system may also include a memory to record the status information. The dosage distribution system may also send notifications to an authorized user, e.g., a patient or physician, to remind the patient or record the taking of the prescribed medicine. For example, if the container of the dosage distribution system has not been emptied or opened after a predetermined period of time, the dosage distribution system may send out a reminder or alert to the patient or physician. In embodiments, the dosage distribution system may send the reminder to an authorized individual, e.g., a physician, parent, or caregiver.

In embodiments, the dosage distribution system has a sensor for processing signals. For example, the sensor may detect the contents of each container. In embodiments, when a sensor detects that one or more dosage forms (e.g., tablets or capsules) are taken from the container the sensor records the information and/or notifies an authorized user. For example, the information may be sent to a connected communication device, which directly notifies the user, or through the internet-based service send a notification to multiple people who are authorized to receive the information.

In embodiments methods of preventing and/or treating contrast-induced acute kidney injury includes administering to a patient in need thereof a dosage distribution system which includes a container for storing a dosage form and a sensor for processing signals wherein the dosage form includes trimetazidine or a pharmaceutically acceptable salt thereof. The duration of action and therefore efficacy may be dependent on plasma half-life of the medicine. Since efficacy is often dependent on sufficient and consistent exposure administration may require frequent maintenance dosing. For example, trimetazidine or a pharmaceutically acceptable salt thereof may be used with the dosage distribution system to administer a fixed dose, at regular intervals, to achieve therapeutic efficacy. Advantageously disclosed herein are methods of preventing contrast-induced acute kidney injury by administration of trimetazidine or pharmaceutically acceptable salt thereof. For example, in embodiments, methods of treating a contrast-induced acute kidney injury are provided which include administering to a patient in need thereof a pharmaceutical composition including about 20 mg to about 75 mg of trimetazidine or pharmaceutically acceptable salt thereof using a dosage distribution system.

In embodiments, the patient is administered trimetazidine or a pharmaceutically acceptable salt thereof prior to undergoing a cardiac procedure. For example, the patient may be administered trimetazidine or a pharmaceutically acceptable salt thereof for more than, e.g., 24, 48, or 72 hours, prior to undergoing a cardiac procedure. In other examples, the patient in need thereof is administered trimetazidine or a pharmaceutically acceptable salt thereof for at least 96 hours prior to undergoing a cardiac procedure.

In embodiments, the dosage distribution system includes a container for storing a dosage form. The container may include a body with multiple cells for containing a dosage form. A sensor for processing signals may be located in the container, e.g., at the bottom of each cell or in the cap, to detect the presence or absence of dosage forms in each cell or the opening of the container. In embodiments, the sensor may process signals and communicate information to a wireless device. In embodiments, the dosage distribution system may also include a microprocessor unit and memory for controlling the sensor and/or a communication circuit. In embodiments, the dosage distribution system provides methods for actively monitoring the status of the container. In embodiments, the dosage distribution system detects motion of the container.

In embodiments the dosage distribution system includes a container with multiple cells to organize daily medicine or different dosages to be taken at different times. The dosage distribution system may include sensors in each cell to detect if any are empty or includes one or more dosage forms. In embodiments the dosage distribution system may communicate with, e.g., a smart phone or a network, through a wireless connection, e.g., Bluetooth or Wi-Fi. In embodiments the dosage distribution system may keep a record of when dosage forms, e.g., tablets or capsules, are taken from a container, and provide a summary of compliance based on a predetermined dosage regime. In embodiments, the dosage distribution system sends a reminder to a recipient, e.g., a patient, caregiver, etc., to take the medicine at an appropriate time.

In embodiments, the dosage distribution system is used in connection with determining efficacy of treatment. For example, signals collected by the dosage distribution system are compared with at least one level of serum creatinine, troponin T or creatine kinase. In other examples, signals collected by the dosage distribution system are compared with levels of trimetazidine or a pharmaceutically acceptable salt thereof in the blood of the patient.

In embodiments, trimetazidine or pharmaceutically acceptable salt thereof may be administered once daily. In embodiments, trimetazidine or pharmaceutically acceptable salt thereof may be administered twice daily. In embodiments, trimetazidine or pharmaceutically acceptable salt thereof may be administered three times daily. In embodiments, trimetazidine or pharmaceutically acceptable salt thereof may be administered four times daily. In embodiments, an inhibitor of fatty acid oxidation may be administered once, two, three or four times daily.

In embodiments, the patient may be administered an inhibitor of fatty acid oxidation for more than, e.g., 30, 45, 60, 75, or 90 days, after undergoing a cardiac procedure. In embodiments, the patient may be administered trimetazidine or a pharmaceutically acceptable salt thereof for more than, e.g., 30, 45, 60, 75, or 90 days, after undergoing a cardiac procedure. For example, the patient may be administered trimetazidine or a pharmaceutically acceptable salt thereof twice daily for more than 60 days after undergoing a cardiac procedure. In other examples, the patient may be administered trimetazidine or a pharmaceutically acceptable salt thereof twice daily for a total of 90 days.

In embodiments, the patient may be administered trimetazidine or a pharmaceutically acceptable salt thereof three times daily for more than 48 hours prior to undergoing a cardiac procedure and administered trimetazidine or a pharmaceutically acceptable salt thereof twice daily for more than 60 days after undergoing the cardiac procedure. In embodiments, the patient may be administered etomoxir, oxfenicine, perhexiline, mildronate, or ranolazine or a pharmaceutically acceptable salt of any of the preceding three times daily for more than 48 hours prior to undergoing a cardiac procedure and administered trimetazidine or a pharmaceutically acceptable salt thereof twice daily for more than 60 days after undergoing the cardiac procedure.

In embodiments, the dosage distribution system may be used to administer trimetazidine or a pharmaceutically acceptable salt thereof at regular intervals to achieve therapeutic efficacy. The methods include preventing and/or treating contrast-induced acute kidney injury by administering to a patient in need thereof an inhibitor of fatty acid oxidation within 6 hours prior to administration of a contrast agent. In embodiments, methods of preventing and/or treating contrast-induced acute kidney injury include administering to a patient in need thereof an inhibitor of fatty acid oxidation within 5, 4, 3, 2 or 1 hour(s) prior to administration of a contrast agent. In embodiments, methods of preventing and/or treating contrast-induced acute kidney injury include parenterally administering to a patient in need thereof an inhibitor of fatty acid oxidation within 6 hours prior to administration of a contrast agent. In embodiments, methods of preventing and/or treating contrast-induced acute kidney injury include parenterally administering to a patient in need thereof an inhibitor of fatty acid oxidation within 5, 4, 3, 2 or 1 hour(s) prior to administration of a contrast agent. In embodiments, such parenteral administration is intravenous administration.

In embodiments, trimetazidine or pharmaceutically acceptable salt or a pharmaceutically acceptable salt thereof is administered at dosages ranging from about 0.001 mg/kg and about 10 mg/kg of body weight of a patient in need thereof, e.g., from about 0.01 mg/kg to 2.0 mg/kg at least once a day. For example, dosage forms that may be used with the dosage distribution system includes amounts of trimetazidine or a pharmaceutically acceptable salt thereof in the range of about, e.g., 1 mg to 30 mg, 1 mg to 20 mg, 1 mg to 15 mg, 0.01 mg to 10 mg, 0.1 mg to 15 mg, 0.15 mg to 12.5 mg, or 0.2 mg to 10 mg, with doses of 0.1 mg, 0.2 mg, 0.3 mg, 0.4 mg, 0.5 mg, 0.6 mg, 0.7 mg, 0.8 mg, 0.9 mg, 1.5 mg, 1.0 mg, 1.75 mg, 2 mg, 2.5 mg, 2.75 mg, 3 mg, 3.5 mg, 3.75 mg, 4 mg, 4.5 mg, 4.75 mg, 5 mg, 5.5 mg, 6 mg, 6.5 mg, 7 mg, 7.5 mg, 8 mg, 8.5 mg, 9 mg, 10 mg, 11 mg, 12 mg, 15 mg, 20 mg, 25 mg, and 30 mg being specific examples of doses.

Typically, dosages of trimetazidine or pharmaceutically acceptable salt or pharmaceutically acceptable salts thereof are administered one to four times daily to a patient in need thereof. The methods and compositions described herein may provide reduced adverse events and/or increased efficacy. In embodiments, the dosage is about, e.g., 20-100 mg/day, or 21-100 mg/day, or 22-100 mg/day, or 23-100 mg/day, for example 21 mg/day, 42 mg/day, 63 mg/day, 22 mg/day, 44 mg/day, 66 mg/day, 23 mg/day, 46 mg/day, 69 mg/day, 24 mg/day, 48 mg/day, 72 mg/day, 25 mg/day, 50 mg/day or 75 mg/day.

Provided herein are dosing regimens that allow prevention and/or treatment of contrast-induced acute kidney injury with potentially limited or substantially few negative side effects, e.g., Parkinson's disease Parkinson's symptoms, or Parkinson's like symptoms. For example, the dosage distribution system may provide consistent administration of trimetazidine or pharmaceutically acceptable salt thereof such that the patient experiences limited or substantially few negative side effect. One skilled in the art may expect the use and dosage regimes provided herein to cause, or increase the risk of, Parkinsonian symptoms. Accordingly, the methods described herein may provide treatment of a contrast-induced acute kidney injury that may be considered surprising and unexpected. For example, methods are provided herein of preventing and/or treating contrast-induced acute kidney injury in a patient in need thereof that may not cause Parkinson's like symptoms, e.g., tremors, muscle rigidity, slow movement, and impaired balance and coordination.

Trimetazidine or pharmaceutically acceptable salt thereof may be provided as an acid addition salt, a zwitter ion hydrate, zwitter ion anhydrate, dihydrochloride, hydrochloride or hydrobromide salt, or in the form of the zwitter ion monohydrate. Acid addition salts, include but are not limited to, maleic, fumaric, benzoic, ascorbic, succinic, oxalic, bis-methylenesalicylic, methanesulfonic, ethane-disulfonic, acetic, propionic, tartaric, salicylic, citric, gluconic, lactic, malic, mandelic, cinnamic, citraconic, aspartic, stearic, palmitic, itaconic, glycolic, p-amino-benzoic, glutamic, benzene sulfonic or theophylline acetic acid addition salts, as well as the 8-halotheophyllines, for example 8-bromo-theophylline. In embodiments, inorganic acid addition salts, including but not limited to, hydrochloric, hydrobromic, sulfuric, sulfamic, phosphoric or nitric acid addition salts may be used. In embodiments, the trimetazidine may be provided as a dihydrochloride salt. One skilled in the art will readily understand that the amounts of active ingredient in a pharmaceutical composition will depend on the form of trimetazidine provided.

Deuteration of pharmaceuticals to improve pharmacokinetics (PK), pharmacodynamics (PD), and toxicity profiles, has been demonstrated previously with some classes of drugs. Accordingly the use of deuterium-enriched trimetazidine is contemplated and within the scope of the methods and compositions described herein. Deuterium can be incorporated in any position in replace of hydrogen synthetically, according to the synthetic procedures known in the art. For example, deuterium may be incorporated to various positions having an exchangeable proton, such as the amine N—H, via proton-deuterium equilibrium exchange. Thus, deuterium may be incorporated selectively or non-selectively through methods known in the art.

In embodiments, methods of treating a contrast-induced acute kidney injury include administering to a patient in need thereof a pharmaceutical composition including more than about 20 mg to about 75 mg trimetazidine or a pharmaceutically acceptable salt thereof. In embodiments, methods of treating a contrast-induced acute kidney injury include administering to a patient in need thereof a pharmaceutical composition including more than about 20 mg trimetazidine or a pharmaceutically acceptable salt thereof. In embodiments, methods of treating a contrast-induced acute kidney injury include administering to a patient in need thereof a pharmaceutical composition including more than about 35 mg trimetazidine or a pharmaceutically acceptable salt thereof.

In embodiments, the provided are methods of preventing and/or treating contrast-induced acute kidney injury which includes administering to a patient in need thereof a first dosage regimen and a second dosage regimen of an inhibitor of fatty acid oxidation. In embodiments, the provided are methods of preventing contrast-induced acute kidney injury which includes administering to a patient in need thereof a first dosage regimen and a second dosage regimen of trimetazidine or pharmaceutically acceptable salt thereof.

In embodiments, the dosage distribution system may be used to administer a first dosage regimen and a second dosage regimen of trimetazidine or a pharmaceutically acceptable salt thereof to achieve therapeutic efficacy. For example, the dosage distribution system may used to administer a first dosage regimen and a second dosage regimen that have different dosages of trimetazidine or a pharmaceutically acceptable salt thereof, different time intervals between dosages, or a combination of both.

In embodiments, the provided are methods of preventing and/or treating contrast-induced acute kidney injury which includes administering to a patient in need thereof a first dosage regimen and a second dosage regimen of one or more of trimetazidine, etomoxir, oxfenicine, perhexiline, mildronate, or ranolazine or a pharmaceutically acceptable salt of any of the preceding. In embodiments, provided are methods of preventing and/or treating contrast-induced acute kidney injury which includes administering to a patient in need thereof a first dosage regimen of trimetazidine or a pharmaceutically acceptable salt thereof and a second dosage regimen of one or more of trimetazidine, etomoxir, oxfenicine, perhexiline, mildronate, or ranolazine or a pharmaceutically acceptable salt of any of the preceding.

For example, the first dosage regimen may be a loading or initiation dosage to achieve a specific exposure of trimetazidine alone or in combination with one or more of etomoxir, oxfenicine, perhexiline, mildronate, or ranolazine in the patient. The loading or initiation dosage may begin prior to a contrast procedure and provide sufficient exposure to prevent contrast-induced acute injury. In embodiments, the loading dosage may be achieved by a bolus injection of trimetazidine or pharmaceutically acceptable salt thereof alone or in combination with one or more of etomoxir, oxfenicine, perhexiline, mildronate, or ranolazine or a pharmaceutically acceptable salt of any of the proceeding. In embodiments, the loading dosage may be achieved by administering trimetazidine or pharmaceutically acceptable salt thereof alone or in combination with one or more of etomoxir, oxfenicine, perhexiline, mildronate, or ranolazine or a pharmaceutically acceptable salt of any of the proceeding over a period of time before a contrast procedure. For example, trimetazidine or pharmaceutically acceptable salt thereof alone or in combination with one or more of etomoxir, oxfenicine, perhexiline, mildronate, or ranolazine or a pharmaceutically acceptable salt of any of the proceeding, may be administered 6 hours, 12, or 24 hours prior to a contrast procedure. In embodiments, trimetazidine or pharmaceutically acceptable salt thereof, alone or in combination with one or more of etomoxir, oxfenicine, perhexiline, mildronate, or ranolazine or a pharmaceutically acceptable salt of any of the preceding, may be administered within 6 hours prior to administration of a contrast agent. In embodiments, trimetazidine or pharmaceutically acceptable salt thereof alone or in combination with one or more of etomoxir, oxfenicine, perhexiline, mildronate, or ranolazine or a pharmaceutically acceptable salt of any of the preceding, may be administered within 5 hours prior to administration of a contrast agent. In embodiments, trimetazidine or pharmaceutically acceptable salt thereof, alone or in combination with one or more of etomoxir, oxfenicine, perhexiline, mildronate, or ranolazine or a pharmaceutically acceptable salt of any of the preceding, may be administered within 4 hours prior to administration of a contrast agent. In embodiments, trimetazidine or pharmaceutically acceptable salt thereof alone or in combination with one or more of etomoxir, oxfenicine, perhexiline, mildronate, or ranolazine or a pharmaceutically acceptable salt of any of the preceding, may be administered within 3 hours prior to administration of a contrast agent. In embodiments, trimetazidine or pharmaceutically acceptable salt thereof, alone or in combination with one or more of etomoxir, oxfenicine, perhexiline, mildronate, or ranolazine or a pharmaceutically acceptable salt of any of the preceding, may be administered within 2 hours prior to administration of a contrast agent. In embodiments, trimetazidine or pharmaceutically acceptable salt thereof alone or in combination with one or more of etomoxir, oxfenicine, perhexiline, mildronate, or ranolazine or a pharmaceutically acceptable salt of any of the preceding, may be administered within 1 hour prior to administration of a contrast agent. In embodiments, trimetazidine or pharmaceutically acceptable salt thereof alone or in combination with one or more of etomoxir, oxfenicine, perhexiline, mildronate, or ranolazine or a pharmaceutically acceptable salt of any of the preceding, may be administered parenterally. In embodiments, trimetazidine or pharmaceutically acceptable salt thereof alone or in combination with one or more of etomoxir, oxfenicine, perhexiline, mildronate, or ranolazine or a pharmaceutically acceptable salt of any of the preceding, may be administered intravenously. In embodiments, trimetazidine or pharmaceutically acceptable salt thereof etomoxir, oxfenicine, perhexiline, mildronate, or ranolazine may be administered multiple days prior to a contrast procedure, e.g., one, two, three, four, five, six, etc. days prior to a contrast procedure. In embodiments, the loading or initiation dosage regime will be administered three days prior to a contrast procedure.

The second dosage regimen may be a continuation dosage to achieve and/or maintain a specific exposure of trimetazidine, etomoxir, oxfenicine, perhexiline, mildronate, or ranolazine in the patient. The continuation dosage may begin just after a contrast procedure and provide sufficient exposure to prevent contrast-induced acute injury. The continuation dosage may be achieved by administering trimetazidine or pharmaceutically acceptable salt thereof alone or in combination with one or more of etomoxir, oxfenicine, perhexiline, mildronate, or ranolazine, or a pharmaceutically acceptable salt of any of the preceding over a period of time after a contrast procedure. For example, trimetazidine or pharmaceutically acceptable salt thereof alone or in combination with one or more of etomoxir, oxfenicine, perhexiline, mildronate, or ranolazine, or a pharmaceutically acceptable salt of any of the preceding may be administered 6 hours, 12, or 24 hours after a contrast procedure. In embodiments, trimetazidine or pharmaceutically acceptable salt thereof alone or in combination with one or more of etomoxir, oxfenicine, perhexiline, mildronate, or ranolazine, or a pharmaceutically acceptable salt of any of the preceding may be administered multiple days after a contrast procedure, e.g., one, two, three, four, five, six, etc. days after a contrast procedure. In embodiments, the continuation dosage regime will be administered for a period of weeks, e.g., 6, 8, 10 or 12 weeks, after a contrast procedure. In some examples, the continuation dosage will be administered for a period of about 90 days. In some examples, the continuation dosage will be administered for a period of about 87 days. For example, in embodiments, the initiation dosage will be administered for 3 days prior to a contrast procedure and the continuation dosage may be administered for a period of 87 days. Accordingly in some examples, the methods disclosed contemplate the administration of the dosages and profiles described herein for a period of 90 days.

In embodiments, the first dosage regimen and second dosage regimen may be the same or a different dosage of trimetazidine or pharmaceutically acceptable salt thereof alone or in combination with one or more of etomoxir, oxfenicine, perhexiline, mildronate, or ranolazine, or a pharmaceutically acceptable salt of any of the preceding. Thus, the first dosage regimen and second dosage regimen may provide the same or a different profile of trimetazidine, etomoxir, oxfenicine, perhexiline, mildronate, or ranolazine in the patient (e.g., Cₘₐₓ, AUC_{0-∞}) as described herein. In embodiments, the first dosage regimen and second dosage regimen provide the same exposure of trimetazidine, etomoxir, oxfenicine, perhexiline, mildronate, or ranolazine.

In embodiments, the methods and dosage forms include 21 mg to 25 mg, 21 mg to 23 mg, 21 mg to 35 mg, 20.5 mg to 25 mg, 22 mg to 30 mg, 20 to 45 mg, 21 mg to 75 mg, 21 mg to 50 mg, 21 mg to 45 mg, 22 mg to 75 mg, 22 mg to 50 mg, 22 mg to 45 mg, 22 mg to 35 mg, 21 mg to 30 mg, 23 mg to 75 mg, 23 mg to 55 mg, 23 mg to 40 mg, 23 mg to 55 mg, 24 mg to 35 mg, 24 mg to 50 mg, 24 mg to 35 mg trimetazidine or a pharmaceutically acceptable salt thereof.

In embodiments, the methods and dosage forms include 20 mg to 30 mg, 21 mg to 30 mg, 22 mg to 30 mg, 23 mg to 30 mg, 24 mg to 25 mg, 21 mg to 50 mg, 21 mg to 24 mg, 21 mg to 23 mg, or 21 mg to 22 mg trimetazidine or a pharmaceutically acceptable salt thereof.

In embodiments, the methods and dosage forms include, e.g., 20.1 mg, 20.25 mg, 20.5 mg, 21 mg, 21.5 mg, 23 mg, 24 mg, 25 mg, 22 mg, 22.5 mg, 23 mg, 23.5 mg, 24 mg, 24.5 mg, 25.5 mg, 26 mg, 26.5 mg, 27 mg, 27.5 mg, 28 mg, 28.5 mg, 29 mg, 29.5 mg, 30 mg, 30.5 mg, 31 mg, 31.5 mg, 32 mg, 32.5 mg, 33 mg, 33.5 mg, 34 mg, 34.5 mg or 35 mg trimetazidine or a pharmaceutically acceptable salt thereof or amounts that are multiples of such doses. In embodiments, the dosage forms include 21 mg, 22 mg, 23 mg, 24 mg, 25 mg, or 26 mg trimetazidine or a pharmaceutically acceptable salt thereof.

The dosage forms herein may be provided with immediate release, delayed release, extended release, or modified release profiles. In addition, dosage forms herein may be provided for parenteral administration (e.g., intramuscular, intravenous, subcutaneous, intraperitoneal, or intrathecal). In embodiments, dosage forms may be provided with immediate release profile. In embodiments, dosage forms with different drug release profiles may be combined to create a two phase or three-phase release profile. For example, dosage forms may be provided with an immediate release and an extended release profile. In embodiments, pharmaceutical compositions may be provided with an extended release and delayed release profile. Such composition may be provided as pulsatile formulations, multilayer tablets, or capsules containing tablets, beads, granules, etc. Compositions may be prepared using a pharmaceutically acceptable “carrier” composed of materials that are considered safe and effective. The “carrier” includes all components present in the pharmaceutical formulation other than the active ingredient or ingredients. The term “carrier” includes, but is not limited to, diluents, binders, lubricants, disintegrants, fillers, and coating compositions.

In embodiments, the methods and pharmaceutical compositions described herein are administered once, twice, three or four times daily, or every other day. In embodiments, a pharmaceutical composition described herein is provided to the patient in the evening. In embodiments, a pharmaceutical composition described herein is provided to the patient once in the evening and once in the morning.

In embodiments, the dosage distribution system sends a reminder to take a pharmaceutical composition herein, e.g., trimetazidine or a pharmaceutically acceptable salt thereof, three times per day. In embodiments, the dosage distribution system sends a reminder to take a pharmaceutical composition herein, e.g., trimetazidine or a pharmaceutically acceptable salt thereof, two times per day. In embodiments, the dosage distribution system sends a reminder to take a pharmaceutical composition herein, e.g., trimetazidine or a pharmaceutically acceptable salt thereof, for more than 48 hours prior to undergoing a cardiac procedure. In embodiments, the dosage distribution system sends a reminder to take a pharmaceutical composition herein, e.g., trimetazidine or a pharmaceutically acceptable salt thereof, for at least 96 hours prior to undergoing a cardiac procedure. In embodiments, the dosage distribution system sends a reminder to take a pharmaceutical composition herein, e.g., trimetazidine or a pharmaceutically acceptable salt thereof, for more than 60 days after undergoing a cardiac procedure. In embodiments, the dosage distribution system sends a reminder to take a pharmaceutical composition herein, e.g., trimetazidine or a pharmaceutically acceptable salt thereof, once daily for more than 60 days after undergoing a cardiac procedure. In embodiments, the dosage distribution system sends a reminder to take a pharmaceutical composition herein, e.g., trimetazidine or a pharmaceutically acceptable salt thereof, once daily for a total of 90 days. In embodiments, the dosage distribution system sends a reminder to take a pharmaceutical composition herein, e.g., trimetazidine or a pharmaceutically acceptable salt thereof, for more than 48 hours prior to undergoing a cardiac procedure and twice daily for more than 60 days after undergoing the cardiac procedure. In embodiments, the dosage distribution system sends a reminder to take a pharmaceutical composition herein, e.g., trimetazidine or a pharmaceutically acceptable salt thereof, three times daily for more than 60 days after undergoing a cardiac procedure. In embodiments, the dosage distribution system sends a reminder to take a pharmaceutical composition herein, e.g., trimetazidine or a pharmaceutically acceptable salt thereof, three times daily for a total of 90 days. In embodiments, the dosage distribution system sends a reminder to take a pharmaceutical composition herein, e.g., trimetazidine or a pharmaceutically acceptable salt thereof, three times daily for more than 48 hours prior to undergoing a cardiac procedure and three times daily for more than 60 days after undergoing the cardiac procedure.

In embodiments, the total amount of trimetazidine or pharmaceutically acceptable salt thereof administered to a subject in a 24-hour period is 20 mg to 100 mg. In embodiments, the total amount of trimetazidine or pharmaceutically acceptable salt thereof administered to a subject in a 24-hour period is 20 mg to 80 mg. In embodiments, the total amount of trimetazidine or pharmaceutically acceptable salt thereof administered to a subject in a 24-hour period is more than about 20 mg, 40 mg, or 60 mg. In embodiments, the total amount of trimetazidine or a pharmaceutically acceptable salt thereof administered to a subject in a 24-hour period is more than about 20 mg to 60 mg.

In embodiments, provided herein are methods of preventing and/or treating contrast-induced acute kidney injury by administering to a patient in need thereof an effective amount of trimetazidine or pharmaceutically acceptable salt thereof alone or in combination with one or more of etomoxir, oxfenicine, perhexiline, mildronate, or ranolazine, or a pharmaceutically acceptable salt of any of the preceding. An effective amount or therapeutically effective amount can be a dosage sufficient to treat, inhibit, or alleviate one or more symptoms of a contrast-induced acute kidney injury. For example, an effective amount of trimetazidine may be established by measuring the proportion of patients developing a relative decrease of >=30% in estimated or measured glomerular filtration rate (GFR) after some amount of time (e.g., 15, 30, 60 or 90 days) following the administration of a contrast agent. In other examples, an effective amount of trimetazidine, etomoxir, oxfenicine, perhexiline, mildronate, or ranolazine may be established by measuring the proportion of patients with reduction in or loss of kidney function following the administration of a contrast agent. In embodiments, a reduction or loss of kidney function may be determined by measuring an absolute increase of >=0.3 mg/dL in serum creatinine concentration between baseline and at any time (e.g., up to seven days) following the administration of a contrast agent. In embodiments, a reduction or loss of kidney function may be determined by measuring a relative increase of >=50% in serum creatinine concentration between baseline and at any time (e.g., up to seven days) following the administration of a contrast agent. In embodiments, a reduction or loss of kidney function may be determined by measuring a relative decrease of >=30% in estimated or measured glomerular filtration rate (GFR) between baseline and at any time up (e.g., up to seven days) following the administration of a contrast agent.

In embodiments, provided herein are methods of preventing and/or treating contrast-induced acute kidney injury by administering to a patient in need thereof an effective amount of trimetazidine or pharmaceutically acceptable salt thereof in combination with or before and after administration of contrast media such as barium sulfate, iodine, and gadolinium, anticoagulants such as acetylsalicylic acid, unfractionated heparin, low-molecular weight heparin (enoxaparin), synthetic pentasaccharides/Factor Xa inhibitors (including fondaparinux), dipyridamole, argatroban, and/or bivalirudin, ticlopidine, cilostazol, and anti-platelet agents such as cangrelor, clopidogrel, prasugrel, ticagrelor, aspirin, and/or glycoprotein IIb/IIIa antagonists such as abciximab, eptifibatide and tirofiban.

In embodiments, the dosage distribution system may be used to administer trimetazidine or a pharmaceutically acceptable salt thereof to achieve consistent exposure to the patient. For example, the dosage distribution system may be used to administer a consistent and/or predefined pharmacokinetic (pK) profile of trimetazidine or a pharmaceutically acceptable salt thereof.

In embodiments, provided herein are methods of preventing and/or treating contrast-induced acute kidney injury which include administering to a patient in need thereof trimetazidine or a pharmaceutically acceptable salt thereof wherein the composition provides an in vivo plasma profile having a Cₘₐₓ more than about 65 μg/ml.

In embodiments, the composition provides an in vivo plasma profile having a Cₘₐₓ more than about, e.g., 75 μg/ml, 85 ng/ml, 90 ng/ml, or 100 ng/ml. In embodiments, the composition provides an in vivo plasma profile having a Cₘₐₓ less than about, e.g., 250 μg/ml, 200 μg/ml 150 μg/ml, or 100 μg/ml. In embodiments, provided herein are methods of treating a contrast-induced acute kidney injury including administering to a patient in need thereof a pharmaceutical composition including an active substance, e.g., trimetazidine or pharmaceutically acceptable salt, wherein the composition provides an in vivo plasma profile having a Cₘₐₓ more than about 100 μg/ml.

The effective amount of trimetazidine or pharmaceutically acceptable salt necessary for administration may depend on a number of factors. The effective amount of etomoxir, oxfenicine, perhexiline, mildronate, or ranolazine, or a pharmaceutically acceptable salt of any of the preceding necessary for administration may depend on a number of factors. For example, a dosage sufficient to treat, inhibit, or alleviate one or more symptoms of a contrast-induced acute kidney injury will depend on the actual exposure (AUC) experienced by the patient. Accordingly, in some examples, subjects with renal insufficiency undergoing diagnostic or interventional cardiac procedures that require administration of contrast media may require less inhibitor of fatty acid oxidation to reach an exposure that provides an effective amount. In some examples, subjects with renal insufficiency undergoing diagnostic or interventional cardiac procedures that require administration of contrast media may require less trimetazidine or pharmaceutically acceptable salt thereof to reach an exposure that provides an effective amount. In some examples, subjects with renal insufficiency undergoing diagnostic or interventional cardiac procedures that require administration of contrast media may require less etomoxir, oxfenicine, perhexiline, mildronate, or ranolazine, or a pharmaceutically acceptable salt of any of the preceding to reach an exposure that provides an effective amount.

In embodiments, provided herein are methods of preventing and/or treating contrast-induced acute kidney injury which includes administering to a patient in need thereof trimetazidine or a pharmaceutically acceptable salt thereof wherein the composition provides an in vivo plasma profile having an AUC more than about 500 μg hr/ml.

In embodiments, provided herein are methods of preventing and/or treating a contrast-induced acute kidney injury including administering to a patient in need thereof a pharmaceutical composition wherein the composition provides a consistent in vivo plasma profile having a AUC_{0-∞} of more than about 500 μg·hr/ml

In embodiments, the compositions provide an in vivo plasma profile having a AUC_{0-∞} of more than about, e.g., 550 μg·hr/ml, 600 μg·hr/ml, 650 μg·hr/ml, 850 μg·hr/ml, 800 μg·hr/ml, 750 μg·hr/ml, or 700 μg·hr/ml, 900 μg·hr/ml, 1000 μg·hr/ml, 1250 μg·hr/ml, 1500 μg·hr/ml. 2000 μg·hr/ml, 3000 μg·hr/ml, 4000 μg·hr/ml, or 5000 μg·hr/ml.

In embodiments, the patient in need thereof is administered trimetazidine or a pharmaceutically acceptable salt thereof to maintain a AUC_{0-∞} more than 48 hours prior to undergoing a cardiac procedure. For example, the methods may provide and maintain an in vivo plasma profile having a AUC_{0-∞} of more than about, e.g., 500 μg·hr/ml, 600 μg·hr/ml, 750 μg·hr/ml, or 1000 μg·hr/ml.

In embodiments, the patient in need thereof is administered trimetazidine or a pharmaceutically acceptable salt thereof to maintain a AUC_{0-∞} for more than 30 days after undergoing a cardiac procedure. In embodiments, the patient in need thereof is administered trimetazidine or a pharmaceutically acceptable salt thereof to maintain a AUC_{0-∞} for more than 60 days after undergoing a cardiac procedure. In embodiments, the patient in need thereof is administered trimetazidine or a pharmaceutically acceptable salt thereof to maintain a AUC_{0-∞} for more than 75 days after undergoing a cardiac procedure. In embodiments, the patient in need thereof is administered trimetazidine or a pharmaceutically acceptable salt thereof to maintain a AUC_{0-∞} for more than 90 days after undergoing a cardiac procedure. For example, the methods may provide and maintain an in vivo plasma profile having a AUC_{0-∞} of more than about, e.g., 500 μg·hr/ml, 600 μg·hr/ml, 750 μg·hr/ml, or 1000 μg·hr/ml. In embodiments, the patient in need thereof is administered trimetazidine or a pharmaceutically acceptable salt thereof to maintain a AUC_{0-∞} for a total of 90 days.

In embodiments, methods of treating and/or preventing contrast-induced acute kidney injury include administration of trimetazidine or pharmaceutically acceptable salt, pharmaceutically acceptable salts, derivatives and/or analogues thereof in combination with one or more other active agents. The combination therapies can include administration of the active agent together in the same admixture, or in separate admixtures. In embodiments, the pharmaceutical composition includes two, three, or more active agents.

Unless defined otherwise, all technical and scientific terms used herein have the same meanings as commonly understood by one of skill in the art to which the disclosure herein belongs.

The term “about” or “approximately” as used herein means within an acceptable error range for the particular value as determined by one of ordinary skill in the art, which will depend in part on how the value is measured or determined, i.e., the limitations of the measurement system. For example, “about” can mean within 3 or more than 3 standard deviations, per the practice in the art. Alternatively, “about” can mean a range of up to 20%, preferably up to 10%, more preferably up to 5%, and more preferably still up to 1% of a given value. Alternatively, particularly with respect to biological systems or processes, the term can mean within an order of magnitude, preferably within 5-fold, and more preferably within 2-fold, of a value.

As used herein, the term “treating” or “treatment” refers to alleviating, attenuating or delaying the appearance of clinical symptoms of a disease or condition in a subject that may be afflicted with or predisposed to the disease or condition, but does not yet experience or display clinical or subclinical symptoms of the disease or condition. In embodiments, treating” or “treatment” may refer to preventing the appearance of clinical symptoms of a disease or condition in a subject that may be afflicted with or predisposed to the disease or condition, but does not yet experience or display clinical or subclinical symptoms of the disease or condition. “Treating” or “treatment” may also refer to inhibiting the disease or condition, e.g., arresting or reducing at least one clinical or subclinical symptom thereof. “Treating” or “treatment” further refers to relieving the disease or condition, e.g., causing regression of the disease or condition or at least one of its clinical or subclinical symptoms. The benefit to a subject to be treated may be statistically significant, mathematically significant, or at least perceptible to the subject and/or the physician. Nonetheless, prophylactic (preventive) and therapeutic treatment are two separate embodiments of the disclosure herein.

“Effective amount” or “therapeutically effective amount” means a dosage sufficient to alleviate one or more symptom of a disorder, disease, or condition being treated, or to otherwise provide a desired pharmacological and/or physiologic effect.

“Pharmaceutically acceptable” refers to molecular entities and compositions that are “generally regarded as safe,” e.g., that are physiologically tolerable and do not typically produce an allergic or similar untoward reaction, such as gastric upset and the like, when administered to a human. In embodiments, this term refers to molecular entities and compositions approved by a regulatory agency of the federal or a state government, as the GRAS list under section 204(s) and 409 of the Federal Food, Drug and Cosmetic Act, that is subject to premarket review and approval by the FDA or similar lists, the U.S. Pharmacopeia or another generally recognized pharmacopeia for use in animals, and more particularly in humans.

“Co-administered with”, “administered in combination with”, “a combination of” or “administered along with” may be used interchangeably and mean that two or more agents are administered in the course of therapy. The agents may be administered together at the same time or separately in spaced apart intervals. The agents may be administered in a single dosage form or in separate dosage forms.

As used herein, the term “prevention” or “preventing” means to administer a composition to a subject or a system at risk for or having a predisposition for one or more symptoms caused by a disease or disorder to facilitate cessation of a particular symptom of the disease or disorder, a reduction or prevention of one or more symptoms of the disease or disorder, a reduction in the severity of the disease or disorder, the complete ablation of the disease or disorder, stabilization or delay of the epileptic or progression of the disease or disorder.

“Prodrug”, as used herein, refers to a pharmacological substance (drug) that is administered to a subject in an inactive (or significantly less active) form. Once administered, the prodrug is metabolized in the body (in vivo) into a compound having the desired pharmacological activity.

“Analog” and “Derivative” are used herein interchangeably and refer to a compound that possesses the same core as the parent compound, but may differ from the parent compound in bond order, the absence or presence of one or more atoms and/or groups of atoms, and combinations thereof. The derivative can differ from the parent compound, for example, in one or more substituents present on the core, which may include one or more atoms, functional groups, or substructures. In general, a derivative can be imagined to be formed, at least theoretically, from the parent compound via chemical and/or physical processes.

“Stereoisomer”, as used herein, refers to isomeric molecules that have the same molecular formula and sequence of bonded atoms (constitution), but which differ in the three dimensional orientations of their atoms in space. Examples of stereoisomers include enantiomers and diastereomers. As used herein, an enantiomer refers to one of the two mirror-image forms of an optically active or chiral molecule. Diastereomers (or diastereoisomers) are stereoisomers that are not enantiomers (non-superimposable mirror images of each other). Chiral molecules contain a chiral center, also referred to as a stereocenter or stereogenic center, which is any point, though not necessarily an atom, in a molecule bearing groups such that an interchanging of any two groups leads to a stereoisomer. In organic compounds, the chiral center is typically a carbon, phosphorus or sulfur atom, though it is also possible for other atoms to be stereocenters in organic and inorganic compounds. A molecule can have multiple stereocenters, giving it many stereoisomers. In compounds whose stereoisomerism is due to tetrahedral stereogenic centers (e.g., tetrahedral carbon), the total number of hypothetically possible stereoisomers will not exceed 2 n, where n is the number of tetrahedral stereocenters. Molecules with symmetry frequently have fewer than the maximum possible number of stereoisomers. A 50:50 mixture of enantiomers is referred to as a racemic mixture. Alternatively, a mixture of enantiomers can be enantiomerically enriched so that one enantiomer is present in an amount greater than 50%. Enantiomers and/or diasteromers can be resolved or separated using techniques known in the art. “Chirality” also includes axial and planar chirality.

The term “pharmaceutically acceptable salt”, as used herein, refers to derivatives of the compounds defined herein, wherein the parent compound is modified by making acid or base salts thereof. Example of pharmaceutically acceptable salts include but are not limited to mineral or organic acid salts of basic residues such as amines; and alkali or organic salts of acidic residues such as carboxylic acids. The pharmaceutically acceptable salts include the conventional non-toxic salts or the quaternary ammonium salts of the parent compound formed, for example, from non-toxic inorganic or organic acids. Such conventional non-toxic salts include those derived from inorganic acids such as hydrochloric, hydrobromic, sulfuric, sulfamic, phosphoric, and nitric acids; and the salts prepared from organic acids such as acetic, propionic, succinic, glycolic, stearic, lactic, malic, tartaric, citric, ascorbic, pamoic, maleic, hydroxymaleic, phenylacetic, glutamic, benzoic, salicylic, sulfanilic, 2-acetoxybenzoic, fumaric, tolunesulfonic, naphthalenesulfonic, methanesulfonic, ethane disulfonic, oxalic, and isethionic salts.

### EXAMPLES

The examples provided herein are included solely for augmenting the disclosure herein and should not be considered to be limiting in any respect.

### Example 1

The following prophetic Example may be used to assess the efficacy of trimetazidine for the prevention of renal and cardiac injury. This multicenter, randomized, double blind, placebo-controlled study may be used to evaluate the efficacy of trimetazidine for the reduction in major adverse cardiac and renal events, which may include cardiovascular death, non-fatal myocardial infarction, non-fatal stroke, heart failure, need for renal replacement therapy, hospitalization for cardiac, renal, and other reasons, sustained reduction in eGFR of ≥20%, and prolonged or re-hospitalization. This study may also be used to evaluate reduction in the incidence (i.e., prevention of contrast-induced acute kidney injury (CI-AKI). Contrast-induced acute kidney injury may be determined by an increase in serum creatinine (SCr) from baseline of ≥0.3 mg/dL within 48 hours of contrast procedure; or Increase in SCr to ≥1.5 times baseline, which is known or presumed to have occurred within the prior 7 days. Serum concentration of creatinine may also be measured 48 hours, 4 days, and 7 days from time of contrast procedure. Additionally this study may be used to evaluate efficacy of trimetazidine for prevention of cardiac injury in unstable angina (UA) subjects. Reduction in cardiac injury may be determined by cardiac biomarkers in the treatment groups compared to placebo. For example, Plasma concentrations of CK-MB 24 hours following the start of the PCI procedure, a rise in troponin T of at least 5× the upper limit of normal along with clinical signs of ischemia and/or plasma concentrations of troponin. Treatments with trimetazidine at 20 mg, 22.5 mg, 25 mg, 30 mg and/or 33 mg given once, twice or three times daily may be evaluated and compared to placebo. Each subject may receive a dose of either study drug or placebo according to the dosing schedule for a specified period of time, e.g., 30 days, 45 days or 90 days.

### Example 2

The following prophetic Example may be used to assess the impact of trimetazidine on the prevention of contrast induced acute kidney injury in high-risk patients undergoing percutaneous coronary intervention or peripheral transluminal angioplasty. This multicenter, randomized, double blind, placebo-controlled study may be used to evaluate the efficacy of trimetazidine in reducing the incidence of CI-AKI among high-risk patients undergoing planned percutaneous coronary intervention (PCI) or peripheral transluminal angioplasty (PTA). In addition, this study may be used to determine the safety of trimetazidine in reducing the incidence of adverse clinical events (death, RRT, MI, stroke, clinically driven revascularization or major adverse cardiovascular events) at 30 days among high-risk patients undergoing planned PCI or PTA. This study may also be optionally used to determine the safety of trimetazidine in reducing the incidence of adverse clinical events (death, RRT, MI, stroke, clinically driven revascularization or major adverse cardiovascular events) at, e.g., 90 days among high-risk patients undergoing planned PCI or PTA. In addition, this study may optionally be used to determine the safety of trimetazidine in reducing the incidence of adverse clinical events (death, RRT, MI, stroke, clinically driven revascularization or major adverse cardiovascular events) at one year among high-risk patients undergoing planned PCI or PTA.

The primary efficacy endpoint of this study may include the incidence of CI-AKI at 72 hours post contrast media exposure. CI-AKI may be defined as an increase in serum creatinine of more than 0.5 mg/dl (44.2 μmol/L) or a percentage increase in serum creatinine equal to or greater than 50% from the baseline blood draw prior to contrast media exposure. The primary safety endpoint may include the composite occurrence of adverse clinical events at 30 days including death, RRT, MI, stroke, clinically driven revascularization or major adverse cardiovascular events. The primary safety endpoint may optionally include the composite occurrence of adverse clinical events at 90 days including death, RRT, MI, stroke, clinically driven revascularization or major adverse cardiovascular events. In addition, the primary safety endpoint may optionally include the composite occurrence of adverse clinical events at one year including death, RRT, MI, stroke, clinically driven revascularization or major adverse cardiovascular events.

Secondary endpoints may include one or more of the following clinical events within 30 days: All-cause mortality; myocardial infarction; dialysis-dependent renal failure; unplanned re-hospitalization; repeat coronary revascularization of the target lesion; major bleeding (not related to coronary bypass procedures); major adverse cardiovascular events and stroke. Additional secondary endpoint may include the following laboratory-based metrics at 72 hours: percent of subjects with SCr increase of >0.5 mg/dl (44.2 μmol); percent of subjects with SCr increase of >25%; percent of subjects with SCr increase >100%; percent of subjects with SCr increase >200%; and mean change in eGFR.

Those skilled in the art will recognize, or be able to ascertain using no more than routine experimentation, many equivalents to the embodiments and examples described herein. Such equivalents are intended to be encompassed by the claims.

What is claimed is:

1. A method of preventing and/or treating contrast-induced acute kidney injury comprising administering to a patient in need thereof a dosage distribution system comprising a container for storing a dosage form and a sensor for processing signals wherein the dosage form comprises trimetazidine or a pharmaceutically acceptable salt thereof.

2. The method of claim 1, wherein the dosage distribution system comprises a communication circuit for wireless communication with an external device

3. The method of claim 1, wherein the dosage distribution system comprises memory for recording information from the sensor

4. The method of claim 1, wherein the sensor detects the presence or absence of the dosage form in the container.

5. The method of claim 1, wherein the sensor detects an opening or closing of the container.

6. The method of claim 1, wherein the dosage distribution system detects motion of the container.

7. The method of claim 1, wherein dosage distribution system sends a reminder to take trimetazidine or a pharmaceutically acceptable salt thereof three times per day.

8. The method of claim 1, wherein dosage distribution system sends a reminder to take trimetazidine or a pharmaceutically acceptable salt thereof two times per day.

9. The method of claim 1, wherein the sensor detects and records when trimetazidine or a pharmaceutically acceptable salt thereof is taken from the container.

10. The method of claim 1, wherein the signals collected by the dosage distribution system are compared with at least one level of serum creatinine, troponin T or creatine kinase.

11. The method of claim 1, wherein the signals collected by the dosage distribution system are compared with levels of trimetazidine or a pharmaceutically acceptable salt thereof in the blood of the patient.

12. The method of claim 1, wherein the patient in need thereof is undergoing a cardiac procedure that requires administration of contrast media for imaging.

13. The method of claim 1, wherein dosage distribution system sends a reminder to take trimetazidine or a pharmaceutically acceptable salt thereof for more than 48 hours prior to undergoing a cardiac procedure.

14. The method of claim 1, wherein dosage distribution system sends a reminder to take trimetazidine or a pharmaceutically acceptable salt thereof for at least 96 hours prior to undergoing a cardiac procedure.

15. The method of claim 1, wherein dosage distribution system sends a reminder to take trimetazidine or a pharmaceutically acceptable salt thereof for more than 60 days after undergoing a cardiac procedure.

16. The method of claim 1, wherein dosage distribution system sends a reminder to take trimetazidine or a pharmaceutically acceptable salt thereof once daily for more than 60 days after undergoing a cardiac procedure.

17. The method of claim 1, wherein dosage distribution system sends a reminder to take trimetazidine or a pharmaceutically acceptable salt thereof once daily for a total of 90 days.

18. The method of claim 1, wherein dosage distribution system sends a reminder to take trimetazidine or a pharmaceutically acceptable salt thereof once daily for more than 48 hours prior to undergoing a cardiac procedure and twice daily for more than 60 days after undergoing the cardiac procedure.

19. The method of claim 1, wherein dosage distribution system sends a reminder to take trimetazidine or a pharmaceutically acceptable salt thereof twice daily for more than 60 days after undergoing a cardiac procedure.

20. The method of claim 1, wherein dosage distribution system sends a reminder to take trimetazidine or a pharmaceutically acceptable salt thereof twice daily for a total of 90 days.

21. The method of claim 1, wherein dosage distribution system sends a reminder to take trimetazidine or a pharmaceutically acceptable salt thereof three times daily for more than 48 hours prior to undergoing a cardiac procedure and twice daily for more than 60 days after undergoing the cardiac procedure.

22. The method of claim 1, wherein dosage distribution system sends a reminder to take trimetazidine or a pharmaceutically acceptable salt thereof three times daily for more than 60 days after undergoing a cardiac procedure.

23. The method of claim 1, wherein dosage distribution system sends a reminder to take trimetazidine or a pharmaceutically acceptable salt thereof three times daily for a total of 90 days.

24. The method of claim 1, wherein dosage distribution system sends a reminder to take trimetazidine or a pharmaceutically acceptable salt thereof three times daily for more than 48 hours prior to undergoing a cardiac procedure and three times daily for more than 60 days after undergoing the cardiac procedure.

25. The method of claim 1, wherein the dosage form comprises trimetazidine or a pharmaceutically acceptable salt thereof and is administered for up to one year after undergoing a cardiac procedure.

26. The method of claim 1, wherein the dosage form comprises trimetazidine or pharmaceutically acceptable salt thereof as an immediate release tablet.

27. The method of claim 1, wherein the dosage form comprises immediate release tablets with between 10 mg to 100 mg trimetazidine or pharmaceutically acceptable salt thereof.
